(19) United States
(12) Patent Application Publication
(10) Pub. No.: US 2025/0199498 A1
(43) Pub. Date: Jun. 19, 2025

(54) PROCESS-ENGINEERING PLANT MODULE AND METHOD FOR CONTROLLING A PROCESS-ENGINEERING PLANT MODULE

(71) Applicant: Siemens Aktiengesellschaft, München (DE)

(72) Inventors: Jan Christoph Wehrstedt, München (DE); Christoph Wincheringer, München (DE); Harald Held, Bockhorn (DE); Alexander Jastram, München (DE); Dominik Haager, Bruckmühl (DE); Robert Niklas Kunzelmann, München (DE); Thomas Baudisch, Schondorf am Ammersee (DE)

(21) Appl. No.: 18/849,586

(22) PCT Filed: Mar. 20, 2023
(86) PCT No.: PCT/EP2023/057010

§ 371 Date: Sep. 23, 2024

(30) Foreign Application Priority Data
Mar. 31, 2022 (EP) 22165902.2

(51) Int. Cl. G05B 19/05

(52) U.S. Cl. CPC G05B 19/05; G05B 2219/13185

(57) ABSTRACT

A process-engineering plant module is provided including plant components configured to carry out a process-engineering process, wherein a first chemical substance is changed, a sensor, configured to detect a sensor value of a physical quantity of the first chemical substance in a plant component, a simulation unit configured to simulate a chemical reaction, a temperature change and/or a mass transfer for a second chemical substance in a computer-aided manner as a function of the sensor value, wherein the first chemical substance and the second chemical substance are the same or different, and to output a simulated temperature value and a simulated mole fraction of the second chemical substance, and a control unit, which is configured to control actuators of the plant components. A hybrid commissioning of a process-engineering plant, in which a computer-aided simulation of the process-engineering process is carried out in parallel with a cold-commissioning is also provided.

### CROSS-REFERENCE TO RELATED APPLICATIONS

This application is a national stage of PCT Application No. PCT/EP2023/057010, having a filing date of Mar. 20, 2023, claiming priority to EP 22165902.2, having a filing date of Mar. 31, 2022, the entire contents both of which are hereby incorporated by reference.

### FIELD OF TECHNOLOGY

The following relates to a process-engineering plant module and a method for controlling plant components of a process-engineering plant module by a computer-aided simulation of the process-engineering process, and a computer program product.

### BACKGROUND

Before process-engineering plants are taken into productive use, they are put into operation in order to test their functionality and to prevent errors. This involves virtual commissioning, in which automation of the plant is validated by a computer-aided simulation of the technical process. Usually so-called cold-commissioning is simulated. Cold-commissioning here refers to a functional test based on water or other chemically inert substances, instead of the chemical substances actually intended for the process. The technical process is usually not simulated, in order to retain the real-time capability of the simulation and to keep the complexity of the modeling low. Subsequently, real commissioning takes place. In this case cold-commissioning in the real plant is usually also considered, i.e., the plant is tested with harmless inert substances instead of with the real media. This allows the flow controls and other functionalities of the plant to be configured and validated before switching to the real operating conditions and media. However, the commissioning of the process-engineering plant does not therefore take place under real conditions, i.e., in particular the chemical reactions are not taken into account and so the plant cannot be tested in advance with the intended chemical substances.

### SUMMARY

An aspect of embodiments of the invention enable commissioning of a process-engineering plant in which the process-engineering process is also validated.

A first aspect of embodiments of the invention relates to a process-engineering plant module comprising:
a. plant components which are configured to carry out a process-engineering process, wherein a first chemical substance is changed in the process-engineering process,
b. at least one sensor which is configured to detect a sensor value of a physical variable of the first chemical substance in a plant component,
c. a simulation unit which is configured
   to simulate a chemical reaction, a temperature change and/or a mass transfer of the process-engineering process for a second chemical substance as a function of the sensor value in a computer-aided manner, wherein the first chemical substance and the second chemical substance are the same or different, and
   to output a simulated temperature value and a simulated mole fraction of the second chemical substance,
and
d. a control unit which is configured to control actuators of the plant components as a function of the simulated temperature value, the simulated mole fraction and the detected sensor value.

A second aspect of embodiments of the invention relates to a method for controlling plant components of a process-engineering plant module, having the method steps:
a. detection of a sensor value of a physical variable of a first chemical substance in a plant component of the process-engineering plant module, wherein the first chemical substance is changed in a process-engineering process running in the plant components of the process-engineering plant module,
b. computer-aided simulation of a chemical reaction, a temperature change and/or a mass transfer of the process-engineering process for a second chemical substance as a function of the flow value, wherein the first chemical substance and the second chemical substance are the same or different,
c. output of a simulated temperature value and a simulated mole fraction of the second chemical substance,
and
d. control of actuators of the plant components as a function of the simulated temperature value, the simulated mole fraction and the detected sensor value.

The process-engineering plant module can in particular also be referred to as a plant module in the process industry. A process-engineering plant module can be coupled to at least one further process-engineering plant module to form a process-engineering plant. However, a process-engineering plant module can also itself be regarded as a process-engineering plant comprising a plurality of plant components, also referred to as plant modules. In other words, a process-engineering plant can likewise be regarded as a plant module.

A process-engineering process can run in the process-engineering plant module, in which a chemical substance, i.e., an element, a compound or a mixture, which can be present as a liquid, solid or gas, is changed. A change in a chemical substance in a process-engineering process can be understood as a transformation of a substance, i.e., a change in the chemical substance in respect of composition, nature or properties. In the case of cold-commissioning, e.g., during commissioning of a process-engineering plant, the chemical substance is usually water, or nitrogen in the case of gases.

A computer-aided simulation of the process-engineering process is carried out by a computer-aided simulation model of the process-engineering process, wherein the simulation model is configured to map at least one chemical reaction, a mixture, a temperature change and/or a mass transfer of the process-engineering process. In this case in particular consideration is given to the at least one chemical substance which is intended for the process-engineering plant module/process engineering plant after it is commissioned. The simulation model can also be referred to as a digital twin of the process-engineering process.

Embodiments of the invention enable hybrid commissioning of a process-engineering plant, in which a computer-aided simulation of the process-engineering process or of at least one method step or aspect of the process-engineering process is carried out in parallel to cold-commissioning. The simulation results can then be used to control the plant. Hybrid commissioning means that some of the method steps can be replaced by a simulation in the real plant during cold-commissioning. In other words, during commissioning a simulated sensor value can be taken for control of the plant module instead of a real sensor value. In an embodiment, only a process step that is important for the commissioning of the plant module is virtualized. To this end the plant module may be configured so that it comprises an edge device on which steps of the process-engineering process can be simulated in parallel to cold-commissioning. Thus, an automation can be supplemented by signals from the simulation, in order to validate the control of the plant module under realistic conditions.

For example, a flow controller, designed to keep the concentration of a reactant in a reaction vessel constant, can be validated in the real plant during cold-commissioning, by simulating the reaction, temperature change, mixture of chemical substances and/or change in the quantity of substance in the reactor, even though this reaction does not in reality take place during cold-commissioning.

In addition, an embodiment of the invention enables a process-engineering plant module to be operated, wherein values from the simulation can be used for control. For example, virtual sensor values can be used for control of the process-engineering plant module. Thus the accuracy of control and/or the operating safety can be improved.

In an embodiment of the invention the first chemical substance can be chemically inert.

In an embodiment, the first chemical substance can be water and/or nitrogen. Thus cold-commissioning can be carried out in the real plant components, in order for example to test mechanical functionalities of the plant module.

In an embodiment of the invention the sensor can be designed as a virtual sensor and can provide a simulated sensor value.

Thus, additionally or alternatively simulated sensor values can be detected and used for control.

In an embodiment of the invention the sensor can be a flow sensor or a level sensor.

In an embodiment of the invention control commands of the control unit can be taken into account during the computer-aided simulation of the process-engineering process.

This means that the simulation can be adjusted by the current control commands. This enables a simulation to be carried out in parallel to the real cold-commissioning.

In an embodiment of the invention the computer-aided simulation can be carried out in real time and in parallel to the process-engineering process.

A further aspect of an embodiment of the invention relates to a process-engineering plant comprising at least two inventive process-engineering plant modules and a higher-level control unit, wherein the respective control units of the process-engineering plant modules are coupled to one another by the higher-level control unit and wherein the respective simulation units of the process-engineering plant modules are coupled to one another.

Alternatively, the process-engineering plant can comprise only a simulation unit, which can be realized as an edge device or in the cloud.

In an embodiment of the process-engineering plant the respective simulation units of the process-engineering plant modules can be coupled to one another by a co-simulation environment and a first simulated temperature value output by the first simulation unit and a first simulated mole fraction can be used as input data for the second simulation unit.

An aspect of an embodiment of the invention further relates to a computer program product (non-transitory computer readable storage medium having instructions, which when executed by a processor, perform actions) which can be loaded directly into a programmable computer, comprising program code parts, which during the execution of the program by a computer cause the computer to execute the steps of an inventive method.

A computer program product can for example be provided or supplied on a storage medium, for example a memory card, USB stick, CD-ROM, DVD, a non-transitory storage medium or also in the form of a downloadable file from a server in a network.

### BRIEF DESCRIPTION

Some of the embodiments will be described in detail, with reference to the following figures, wherein like designations denote like members, wherein

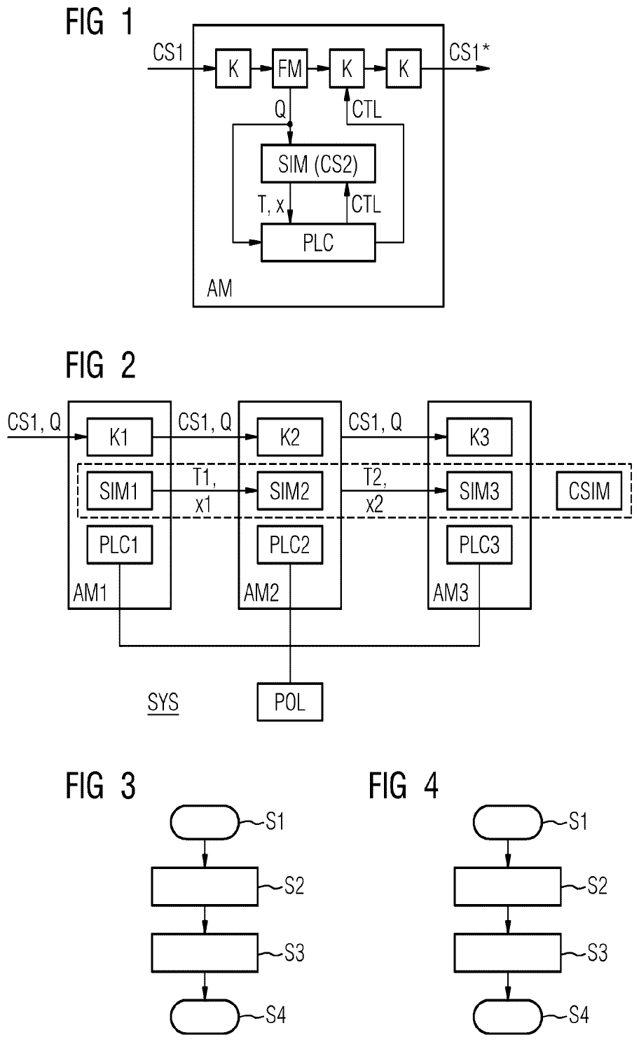

FIG. 1: shows an exemplary embodiment of a process-engineering plant module;

FIG. 2: shows an exemplary embodiment of a process-engineering plant;

FIG. 3: shows a first exemplary embodiment of a method for controlling plant components of a process-engineering plant module; and

FIG. 4: shows a second exemplary embodiment of a method for controlling plant components of a process-engineering plant module.

Parts corresponding to one another are provided with the same reference characters in all figures.

### DETAILED DESCRIPTION

In embodiments, the following exemplary embodiments show only exemplary possible implementations of how in particular such implementations of the inventive teaching could look, since it is impossible and also not expedient or necessary for the understanding of the invention to mention all these possible implementations.

In particular, a (relevant) person skilled in the art, with knowledge of the method claim(s), is of course aware of all possibilities for realizing products or possibilities for implementation that are customary in the conventional art, so that in particular there is no need for a separate disclosure in the description. In particular, these common realization variants known to the person skilled in the art can be realized exclusively using hardware (components) or exclusively using software (components). Alternatively and/or additionally, the person skilled in the art can, within the scope of his or her professional skills, select any inventive combinations of hardware (components) and software (components) in order to implement inventive realization variants.

FIG. 1 shows an exemplary embodiment of an inventive process-engineering plant module AM. Using the process-engineering plant module, hybrid virtual commissioning can in embodiments be realized, wherein cold-commissioning is combined with a computer-aided simulation of the chemical reaction, the temperature change, the mixture of substances and/or the mass transfer. For this, the process-engineering plant module AM may in embodiments befitted with an edge device, on which the process-engineering process can be simulated in parallel to the real cold-commissioning. Alternatively, the computer-aided simulation can also be carried out in the cloud. Thus for example not only flows but also mass transfer, temperature change and chemical reactions can be taken into account and validated during cold-commissioning.

The process-engineering plant module AM in embodiments comprises software and hardware components. The process-engineering plant module AM comprises a plurality of (hardware) plant components K, such as for example chemical reactors, which are connected to one another via lines or pipes (not shown), so that a mass transfer can be realized between the plant components K. The process-engineering plant module AM is therefore configured to carry out a process-engineering process and/or cold-commissioning, wherein a first chemical substance CS1 is changed. The process-engineering process can for example change a property of the first chemical substance CS1, such as for example a temperature change, so that a changed chemical substance CS1* is output.

The process-engineering plant module AM further comprises at least one sensor FM, such as for example a flow meter or a level sensor, a simulation unit SIM and a control unit PLC. The sensor FM can in embodiments also be located outside the process-engineering plant module AM, for example in a coupled plant module, or the sensor can merely be coupled to the process-engineering plant module AM, wherein sensor data is provided via an input unit of the process-engineering plant module.

The control unit PLC is in embodiments the physical control unit of the plant module AM, i.e., the control unit actuates the actuators of the plant components K. In embodiments, the control unit may be a programmable logic controller (PLC). In the architecture the actuator and sensor variables that serve the mass transfer process may be connected directly to the real sensors in embodiments. The signals such as concentrations, temperature, etc., may, for example, come from the simulation in embodiments.

The at least one sensor FM is configured to measure a sensor value of a physical variable of the first chemical substance CS1, such as for example flow, temperature, density, pressure, etc. The sensor FM can for example be positioned in a plant component, at the input and/or output of the process-engineering plant module AM or of a plant component K or between two plant components K. For example, the sensor is a flow meter FM which detects a flow value Q of the first chemical substance CS1 in a plant component. Alternatively, the sensor can also be a level sensor which measures a level of the first chemical substance CS1 in a plant component K.

The simulation unit SIM in embodiments also comprises a physical computer/processor. For example, the simulation unit SIM can comprise an edge device, on which a computer-aided simulation of a chemical reaction, a temperature change and/or a mass transfer of the process-engineering process is executed. Alternatively, the computer-aided simulation can also be carried out in the cloud, wherein the simulation data is provided to the simulation unit.

In embodiments, the simulation unit SIM may comprise at least one computer-aided simulation model of the process-engineering process. The simulation unit SIM is configured to simulate parts of the process-engineering process for at least one second chemical substance CS2 as a function of the flow value Q detected by the flow sensor FM in a computer-aided manner by the simulation model and to output a simulated temperature value T and a simulated mole fraction x of the second chemical substance CS2 as a simulation result. The first chemical substance C1 can in this case be different from the second chemical substance CS2 or can be the same. In embodiments, the simulation can be carried out for more than one second chemical substance.

In a first case this can for example be commissioning of the process-engineering plant module, wherein the first chemical substance CS1 is different from the second chemical substance CS2. In this case the first chemical substance CS1 can for example be a chemically inert substance, such as for example water or nitrogen. For example, cold-commissioning is carried out in the plant components K, controlled by the control unit PLC. During the cold-commissioning the first chemical substance CS1 is processed by the plant components instead of the second chemical substance CS2, wherein in embodiments no chemical reaction takes place.

Thus a functional test of the plant module AM is carried out. The second chemical substance CS2 can for example be the chemical substance which is intended for the process-engineering plant module after it is commissioned. By a computer-aided simulation of parts of the process-engineering process a chemical reaction, a temperature change and/or a mass transfer of the second chemical substance CS2 (or of multiple second chemical substances) can for example be simulated in parallel to cold-commissioning that is actually performed. In this case control commands of the control unit are taken into account during the simulation. The computer-aided simulation supplies a temperature value T and substance quantity value x of the second chemical substance CS2 at a particular point in time. The simulated temperature values T and substance quantity value x are transmitted to the control unit PLC of the plant module AM and are taken into account in the control of the plant module. The control unit PLC controls actuators of the plant components K of the plant module as a function of the detected flow value Q, the simulated temperature value T and the substance quantity value x. In embodiments, the simulated values are used for commissioning instead of the real values from the cold-commissioning. Thus the actuator and sensor variables, which serve the cold-commissioning process, are linked directly to the real sensors. The signals such as concentrations, temperature, etc. come from the simulation model. An exchange of data between the simulation unit SIM and the control unit PLC can take place for example via OPC UA. The exchange of the real and simulated sensor values Q, T, x may be in this case, for example, based on a predetermined sampling rate of the control unit PLC.

The second, alternative case can for example be an operation of the process-engineering module AM, wherein the second chemical substance CS2 corresponds to the first chemical substance CS1. This may be the chemical substance which is intended for the plant module AM in embodiments. During operation of the process-engineering module AM at least one part of the process-engineering process can thus be simulated in parallel in a computer-aided manner. The simulated temperature value T and the simulated mole fraction x of the processed chemical substance can be used for the controller PLC of the process-engineering plant module AM instead of corresponding, actually measured values. In addition, by the computer-aided simulation a simulated flow value can be determined and used for the controller PLC, i.e., the flow sensor can be realized as a virtual sensor.

FIG. 2 shows an exemplary embodiment of a process-engineering plant SYS.

In embodiments, the process-engineering plant SYS comprises at least two, for example, a plurality of process-engineering plant modules AM1-AM3, as shown by way of example in FIG. 1. The process-engineering plant modules AM1-AM3 or the plant components K1-K3 thereof are coupled to one another by lines or pipes (not shown), in order to execute a process-engineering process.

The process-engineering plant SYS further comprises a higher-level control unit POL, also referred to as an orchestration unit/orchestration layer (or process orchestration layer (POL)), which couples the respective control units PLC1-PLC3 of the process-engineering plant modules AM1-AM3 to one another. In this way coupled control can be realized.

The respective simulation units SIM1-SIM3 of the process-engineering plant modules AM1-AM3 are coupled to one another in order to exchange simulation data. In embodiments, the simulation units SIM1-SIM3 may be coupled to one another by a co-simulation environment CSIM. Alternatively, the process-engineering plant SYS can merely comprise a simulation unit.

A process-engineering process of a chemical substance CS1 is carried out in the plant modules AM1-AM3. The chemical substance CS1 is processed in the respective plant components K1-K3 of the plant modules AM1-AM3. For example, cold-commissioning is carried out during commissioning of the process-engineering plant SYS. In parallel to the (real) sequence of the process-engineering process or of the cold-commissioning the process-engineering process of a respective plant module AM1-AM3 is simulated in the corresponding simulation units SIM1-SIM3. Respective simulation results of the respective plant modules AM1-AM3 are used as input data for the subsequent plant module. For example, a simulated temperature value T1 and a simulated substance quantity value x1 as output by the first simulation unit SIM1 are used as input for the second simulation unit SIM2. Correspondingly a simulated temperature value T2 and a simulated substance quantity value x2 as output by the second simulation unit SIM2 are used as input for the third simulation unit SIM3, etc.

In this way a process-engineering plant consisting of multiple system modules AM1-AM3 can be put into operation or operated, whereby the physical connections between the plant modules (water, compressed air, etc.) are connected to one another and the controllers are coupled via a controller of the process control layer POL. The virtual values of the digital twin of the process-engineering process such as substances, temperatures and if appropriate densities are coupled via the plant modules AM1-AM3 by a co-simulation approach. This communication of the coupling between modules in co-simulation in embodiments needs to be less powerful than the coupling between the simulation and the process variables in each individual module, since the material transfer between modules through pipes is slower than the chemical reactions and their coupling to the automation.

FIG. 3 shows an exemplary embodiment of a method for controlling plant components of a process-engineering plant module during commissioning of the plant module. In embodiments, the method can at least in part be computer-implemented.

For example, cold-commissioning is carried out for the commissioning of the plant module. In the first step S1 of the method a sensor value of a physical variable, such as for example a flow value or a level value, of a first chemical substance in a plant component of the process-engineering plant module is detected. In embodiments, the first chemical substance may be chemically inert and is merely used for commissioning of the process-engineering plant module. The first chemical substance is changed in a process-engineering process running in the plant components of the process-engineering plant module; for example only a temperature change takes place, but not a chemical reaction.

In the second method step S2 a part of the process-engineering process which is to run in the plant components is simulated in parallel to the cold-commissioning in a computer-aided manner. Consequently, in embodiments the second method step S2 may run in parallel to the first method step S1 and in real time. In this case a chemical reaction, a temperature change and/or a mass transfer of the process-engineering process in at least some of the plant components is simulated for at least one second chemical substance/for second chemical substances as a function of the sensor value, and a simulated temperature value and a simulated mole fraction of the second chemical substance are determined. In embodiments, the second chemical substance may be different from the first chemical substance and may correspond to the chemical substance with which the process-engineering plant module is to be operated after commissioning. The second chemical substance can also be a mixture of substances.

In the next method step S3 the simulated temperature value and the simulated mole fraction of the second chemical substance(s) are output.

In the next method step S4, actuators of the plant components are controlled as a function of the simulated temperature value, the simulated mole fraction and the detected sensor value. The second to fourth method steps S2-S4 can in embodiments be carried out iteratively, wherein control commands to control the actuators of the plant components are taken into account during the simulation in method step S2.

FIG. 4 shows an exemplary embodiment of a method for controlling plant components of a process-engineering plant module during operation of the plant module. To this end the actually running process-engineering process is simulated at least in part during the operation of the plant in parallel in a computer-aided manner.

In the first method step S1 of the method a sensor value, such as for example a flow value, of a chemical substance is detected in a plant component of the process-engineering plant module. The chemical substance is changed in a process-engineering process running in the plant components of the process-engineering plant module.

In the second method step S2 the process-engineering process is simulated in a computer-aided manner in parallel to operation. In this case a chemical reaction, a temperature change and/or a mass transfer of the process-engineering process of the chemical substance are simulated as a function of the measured sensor value and a simulated temperature value and a simulated mole fraction of the chemical substance are determined.

In the next method step S3 the simulated temperature value and the simulated mole fraction of the second chemical substance are output.

In the next method step S4, actuators of the plant components are controlled as a function of the simulated temperature value, the simulated mole fraction and the detected flow value.

Although the present invention has been disclosed in the form of embodiments and variations thereon, it will be understood that numerous additional modifications and variations could be made thereto without departing from the scope of the invention.

For the sake of clarity, it is to be understood that the use of “a” or “an” throughout this application does not exclude a plurality, and “comprising” does not exclude other steps or elements.

1. A process-engineering plant module #AM4 comprising:
a. plant components which are configured to carry out a process-engineering process, wherein a first chemical substance is changed in the process-engineering process,
b. a sensor which is configured to detect a sensor value of a physical variable of the first chemical substance in a plant component,
c. a simulation unit which is configured
to simulate a chemical reaction, a temperature change and/or a mass transfer of the process-engineering process for a second chemical substance in a computer-aided manner as a function of the sensor value, wherein the first chemical substance and the second chemical substance are the same or different,
and
to output a simulated temperature value and a simulated mole fraction of the second chemical substance,
and
d. a control unit which is configured to control actuators of the plant components as a function of the simulated temperature value, the simulated mole fraction and the detected sensor value.

2. The process-engineering plant module as claimed in claim 1,
wherein the first chemical substance is chemically inert.

3. The process-engineering plant module as claimed in claim 1, wherein the sensor is a flow sensor or a level sensor.

4. The process-engineering plant module as claimed in claim 1, wherein the sensor is designed as a virtual sensor and provides a simulated sensor value.

5. The process-engineering plant module as claimed in claim 1, wherein control commands of the control unit are taken into account in the computer-aided simulation of the process-engineering process.

6. The process-engineering plant module as claimed in claim 1, wherein the computer-aided simulation is carried out in real time and parallel to the process-engineering process.

7. A process-engineering plant comprising at least two process-engineering plant modules as claimed in claim 1 and a higher-level control unit, wherein the respective control units of the process-engineering plant modules are coupled to one another by the higher-level control unit and wherein the respective simulation units of the process-engineering plant modules are coupled to one another.

8. The process-engineering plant as claimed in claim 7, wherein the respective simulation units of the process-engineering plant modules are coupled to one another by a co-simulation environment and wherein a first simulated temperature value output by a first simulation unit and a first simulated mole fraction are used as input data for a second simulation unit.

9. A method for controlling plant components of a process-engineering plant module, comprising:
a. detection of a sensor value of a physical variable of a first chemical substance in a plant component of the process-engineering plant module, wherein the first chemical substance is changed in a process-engineering process running in the plant components of the process-engineering plant module,
b. computer-aided simulation of a chemical reaction, a temperature change and/or a mass transfer of the process-engineering process for a second chemical substance as a function of the sensor value, wherein the first chemical substance and the second chemical substance are the same or different,
c. output of a simulated temperature value and a simulated mole fraction of the second chemical substance,
and
d. control of actuators of the plant components as a function of the simulated temperature value, the simulated mole fraction and the detected sensor value.

10. A computer program product, comprising a computer readable hardware storage device having computer readable program code stored therein, said program code executable by a processor of a computer system implement the method in accordance with claim 9.
